(19) United States
(12) Patent Application Publication
(10) Pub. No.: US 2026/0358631 A1
(43) Pub. Date: Nov. 12, 2026

(54) AUGMENTED-REALITY HEADSETS FOR USE IN MEDICAL SETTINGS INCLUDING SURGERY

(71) Applicants: University of Pittsburgh - Of the Commonwealth System of Higher Education, Pittsburgh, PA (US); UPMC, Pittsburgh, PA (US)

(72) Inventors: Edward Gregory Andrews, Pittsburgh, PA (US); Jacob Biehl, Pittsburgh, PA (US); Reetwan Bandyopadhyay, Pittsburgh, PA (US); Sejeal Katiyar, Pittsburgh, PA (US); Garrett Martin, Pittsburgh, PA (US); Samuel Lord, Pleasantville, NY (US); Andrew Daoud, Pittsburgh, PA (US)

(21) Appl. No.: 19/118,482

(22) PCT Filed: Oct. 4, 2023
(86) PCT No.: PCT/US2023/075973

§ 371 Date: Apr. 4, 2025

Related U.S. Application Data
(60) Provisional application No. 63/413,359, filed on Oct. 5, 2022.

(51) Int. Cl. G02B 27/01; A61B 90/00; F21V 21/088; H04N 13/254; H04N 13/296; H04N 13/344; H04N 23/57; H04N 23/74

(52) U.S. Cl. CPC G02B 27/0172; A61B 90/37; F21V 21/088; H04N 13/254; H04N 13/296; H04N 13/344; H04N 23/57; H04N 23/74; A61B 2090/365; G02B 2027/0118

(57) ABSTRACT

A device for use in connection with an augmented reality headset, includes a body, a headset interface system attached to the body to connect the device to the augmented reality headset, and a camera interface attached to the body for connection of a 3D stereoscopic camera to the device.

### CROSS-REFERENCE TO RELATED APPLICATIONS

This application claims benefit of U.S. Provisional Patent Application Ser. No. 63/413,359, filed Oct. 5, 2022, the disclosure of which is incorporated herein by reference.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

Augmented reality (AR) surgical navigation is a burgeoning area of interest in all surgical specialties. Neurological surgery has developed a particular interest in this technology over the past ten years as it has improved to the point to allow intra-operative visualization of complex pathology that can improve surgical efficiency while reducing surgical complications. Using augmented reality, a surgeon is, for example, able to see hidden anatomy that may otherwise be put at risk, such as vessels at risk of perforation which are positioned off the back of an aneurysm or the boundaries of a tumor abutting critical deep grey structures in the brain. The design of leading augmented reality headsets, however, prohibits adoption in the operating room. While these devices are able to overlay patient anatomy and patient pathology, such as tumors, or pre-planned surgical trajectories, such as in spinal pedicle screw placement, they are unable to fully function as an intra-operative device.

It is thus desirable to develop augmented reality devices that are fully functional as intra-operative devices.

### SUMMARY

In one aspect, a device for use in connection with an augmented reality headset, includes a body, a headset interface system attached to the body to connect the device to the augmented reality headset; and a camera interface attached to the body for connection of a camera to the device. In an number of embodiments, the camera is a 3D stereoscopic camera. The camera interface is configured to attach the 3D stereoscopic camera thereto such that the 3D stereoscopic camera is orientable to provide a view of at least a portion of a field of view of a user when the device is in connection with the augmented reality headset.

In a number of embodiments, the device further includes one or more headlights attached to the body. Each of the one or more headlights may, for example, provide at least one 450 lumens. A plurality of headlights may be attached to the body. In a number of embodiments, the device includes a first headlight attached to a first lateral side of the body and a second headlight attached to a second lateral side of the body.

In a number of embodiments of the device, at least one of: (i) at least one of the one or more headlights is configured to controllably emit light of different ranges of wavelengths, and (ii) the device further includes an excitation filter system attached to the body and being controllably positionable to be in optical connection with one of the one or more headlights. At least one of the ranges of wavelengths may, for example, be suitable to enhance the visualization of fluorophoric molecules or the at least one the excitation filter system may, for example, be configured to enhance the visualization of fluorophoric molecules.

The device further includes an excitation filter system in a number of embodiments. The excitation filter system may include at least one filter for each of fluorescein, indocyanine green, and 5-aminoluvelinic acid.

The device may further include an emission filter system attached to the headset which is controllably positionable to be in operative connection with one of one or more lenses of the 3D stereoscopic camera. The emission filter system may, for example, be configured to enhance the visualization of fluorophoric molecules. In a number of embodiments, the emission filter system include multiple filters. The emission filter system may, for example, include at least one filter for each of fluorescein, indocyanine green, and 5-aminoluvelinic acid.

In a number of embodiments, at least one of the 3D stereoscopic camera, the one or more headlights, the excitation filter system, when present, and the emission filter system, is controllable via at least one of manual control, voice activation or a menu of the augmented reality headset, optionally a holographic menu. In a number of embodiments, each of the 3D stereoscopic camera, the one or more headlights, the excitation filter system, when present, and the emission filter system, is controllable via at least one of manual control, voice activation or a menu of the augmented reality headset, optionally a holographic menu.

In general, one or more components such as the 3D stereoscopic camera, the excitation filter system, when present, and the emission filter system may be controlled in at least a semi-automated fashion via, for example, sensing one or more variables such as eye movement.

The 3D stereoscopic camera and the one or more headlights may, for example, be controllable in concert. In a number of embodiments, software-based eye tracking is used to control at least one of the 3D stereoscopic camera and the one or more headlights.

In another aspect, a headset system includes a support system for supporting the headset system upon the head of a user, a display system attached to the support system and comprising a display, a control system including a processor system and a memory system in communicative connection with the processor system, the memory system having stored therein one or more software algorithms executable by the processor system, the control system being configured to provide an augmented reality display via the display system, and a 3D stereoscopic camera in communicative connection with the control system. The 3D stereoscopic camera is attached to the headset system and is orientable to provide a view of at least a portion of a field of view of the user.

The headset system may further include one or more headlights attached to the headset. Each of the one or more headlights provides at least one 450 lumens. In a number of embodiments, the headset system includes a plurality of headlights attached to the support system. The headset system may, for example, include a first headlight attached to a first lateral side of the support system and a second headlight attached to a second lateral side of the support system.

In a number of embodiment of the headset system, at least one of: (i) at least one of the one or more headlights is configured to controllably emit light of different ranges of wavelengths, and (ii) the headset system further includes an excitation filter system attached to the headset which is controllably positionable to be in optical connection with one of the one or more headlights. At least one of the ranges of wavelengths may be suitable to enhance the visualization of fluorophoric molecules, or the at least one the excitation filter system may be configured to enhance the visualization of fluorophoric molecules.

The headset system may, for example, further include an excitation filter system. The excitation filter system may include at least one filter for each of fluorescein, indocyanine green, and 5-aminoluvelinic acid.

In a number of embodiments, the headset system further includes an emission filter system attached to the headset. The emission filter system may, for example, be controllably positionable to be in operative connection with one of one or more lenses of the 3D stereoscopic camera. The emission filter system may be configured to enhance the visualization of fluorophoric molecules. In a number of embodiments, the emission filter system includes multiple filters. The emission filter system may, for example, include at least one filter for each of fluorescein, indocyanine green, and 5-aminoluvelinic acid.

In a number of embodiments, the headset system includes a positioning system attached to the headset system which is configured to adjust the position of the display. The positioning system is configured to move the position of the display so that the user may directly view at least a portion of an environment surrounding the user without obstruction by the display or without viewing the at least a portion of an environment surrounding the user through the display.

In a number of embodiments, at least one of the 3D stereoscopic camera, the one or more headlights, the excitation filter system, the emission filter system, and the positioning system is controllable via at least one of manual control, voice activation or a menu, optionally a holographic menu. In a number of embodiments, each of the 3D stereoscopic camera, the one or more headlights, the excitation filter system, the emission filter system, and the positioning system is controllable via at least one of voice activation or a menu, optionally a holographic menu.

The display of the display system may be an optical see-through display. In a number of embodiments, the 3D stereoscopic camera and the one or more headlights are controllable in concert. Eye-tracking software stored in the memory system may, for example, be used to control the 3D stereoscopic camera and the one or more headlights.

In a number of embodiments, at least one of the 3D stereoscopic camera, the one or more headlights, the excitation filter system, and the emission filter system is an add-on component to a separate augmented reality headset which comprises the support system, the display system, and the control system. The at least one of the 3D stereoscopic camera, the one or more headlights, the excitation filter system, and the emission filter system may, for example, be placed in operative connectivity with the existing augmented reality headset via a USB connection.

In a number of embodiments, the add-on component is a device including a body, a headset interface system attached to the body to connect the device to the separate augmented reality headset, and a camera interface attached to the body for connection of the 3D stereoscopic camera to the device. The camera interface is configured to attach the 3D stereoscopic camera thereto such that the 3D stereoscopic camera is orientable to provide a view of at least a portion of a field of view of the user when the device is in connection with the augmented reality headset.

In another aspect, a method of performing a medical procedure includes providing a headset system as set forth herein. The medical procedure may, for example, be a surgical medical procedure.

In a further aspect, a method of constructing a headset system for use in medical settings, wherein the headset system include an augmented reality headset including a support system for supporting the headset upon the head of a user, a display system attached to the support system and including a display, and a control system comprising a processor system and a memory system in communicative connection with the processor system, wherein the memory system has stored therein one or more software algorithms executable by the processor system, and wherein the control system is configured to provide an augmented reality display upon the display of the display system, includes attaching a 3D stereoscopic camera to the augmented reality system. The 3D stereoscopic camera is orientable to provide a view of at least a portion of a field of view of the user. The method further includes placing the 3D stereoscopic camera in communicative connection with the control system. In a number of embodiments, the method further includes attaching one or more headlights to the headset, wherein each of the one or more headlights provides at least one 450 lumens.

In a number of embodiments of the method, at least one of: (i) at least one of the one or more headlights is configured to controllably emit light of different ranges of wavelengths, and (ii) the method further includes attaching an excitation filter system to the augmented realty headset which us controllably positionable to be in optical connection with one of the one or more headlights. At least one of the ranges of wavelengths may be suitable to enhance the visualization of fluorophoric molecules of the least one the excitation filter system may be configured to enhance the visualization of fluorophoric molecules.

The method may further include attaching an emission filter system to the augmented reality headset which is controllably positionable to be in operative connection with one of one or more lenses of the 3D stereoscopic camera. The emission filter system may, for example, be configured to enhance the visualization of fluorophoric molecules.

In a number of embodiments, the 3D stereoscopic camera is attached to the augmented reality headset by attaching a device comprising a body, a headset interface system attached to the body to connect the device to the augmented reality headset, and a camera interface attached to the body for connection of the 3D stereoscopic camera to the device. The camera interface may be configured to attach the 3D stereoscopic camera thereto such that the 3D stereoscopic camera is orientable to provide a view of at least a portion of a field of view of the user when the device is in connection with the augmented reality headset.

In still a further aspect, an augmented reality headset includes at least one of: one or more headlight systems, a 3D, stereoscopic camera, an excitation filter system, an emission filter system, and a positioning system which is configured to adjust the position of the display system.

The present devices, systems, and methods along with the attributes and attendant advantages thereof, will best be appreciated and understood in view of the following detailed description taken in conjunction with the accompanying drawings.

### BRIEF DESCRIPTION OF THE DRAWINGS

FIG. 1 illustrates an embodiment of a typical architecture for augmented reality software upon which a number of functionalities provided by embodiments of a headset system hereof are overlain.

FIG. 2 illustrates a front view of an embodiment of an augmented reality headset hereof for use in medical procedures including surgery.

FIG. 3 illustrates a side view of an embodiment of an augmented reality headset hereof.

FIG. 4 illustrates a front view of an embodiment of an augmented reality headset hereof including a stereoscopic camera.

FIG. 5 illustrates the augmented reality headset of FIG. 3 including excitation filter systems and emission filter systems.

FIG. 6 illustrates an enlarged view of an excitation filter system for use in connection with an augmented reality headset hereof which includes multiple, different filters.

FIG. 7A illustrates an isometric view of an embodiment of a device hereof providing additional functionality for use in connection with an augmented reality headset hereof for use in medical procedures including surgery.

FIG. 7B illustrates another isometric view of the device of FIG. 7A.

FIG. 7C illustrates front view of the device of FIG. 7A.

FIG. 7D illustrates a side view of the device of FIG. 7A.

FIG. 8A illustrates top view of the device of FIG. 7A with a camera in operative connection therewith.

FIG. 8B illustrates an isometric view of the device of FIG. 7A with a camera in operative connection therewith.

FIG. 8C illustrates a front view of the device of FIG. 7A with a camera in operative connection therewith.

FIG. 8D illustrates a side view of the device of FIG. 7A with a camera in operative connection therewith.

FIG. 8E illustrates another isometric view of the device of FIG. 7A with a camera in operative connection therewith.

FIG. 8F illustrates another isometric view of the device of FIG. 7A with a camera in operative connection therewith.

FIG. 8G illustrates another isometric view of the device of FIG. 7A with a camera in operative connection therewith.

FIG. 9A illustrates an isometric view of an embodiment of a system hereof including the device of FIG. 7A and an augmented reality headset (illustrated schematically) in operative connection therewith (without a 3D stereoscopic camera attached to the device).

FIG. 9B illustrates a top view of the system of FIG. 9A with the camera attached to the device.

FIG. 9C illustrates a front view of the system of FIG. 9A with the camera attached to the device.

FIG. 9D illustrates a side view of the system of FIG. 9A with the camera attached to the device.

FIG. 9E illustrates an isometric view of the system of FIG. 9A wherein the device of FIG. 7A, which includes a stereoscopic camera in operative connection therewith, is disconnected from the augmented reality headset (a portion of which is illustrated schematically and in broken lines in FIG. 9E).

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “a filter system” includes a plurality of such filter systems and equivalents thereof known to those skilled in the art, and so forth, and reference to “the filter system” is a reference to one or more such filter systems and equivalents thereof known to those skilled in the art, and so forth. Recitation of ranges of values herein are merely intended to serve as a shorthand method of referring individually to each separate value falling within the range. Unless otherwise indicated herein, each separate value, as well as intermediate ranges, are incorporated into the specification as if individually recited herein. All methods described herein can be performed in any suitable order unless otherwise indicated herein or otherwise clearly contraindicated by the text.

The terms “electronic circuitry”, “circuitry” or “circuit,” as used herein include, but is not limited to, hardware, firmware, software, or combinations of each to perform a function(s) or an action(s). For example, based on a desired feature or need, a circuit may include a software-controlled microprocessor, discrete logic such as an application specific integrated circuit (ASIC), or other programmed logic device. A circuit may also be fully embodied as software. As used herein, “circuit” is considered synonymous with “logic.” The term “logic”, as used herein includes, but is not limited to, hardware, firmware, software, or combinations of each to perform a function(s) or an action(s), or to cause a function or action from another component. For example, based on a desired application or need, logic may include a software-controlled microprocessor, discrete logic such as an application specific integrated circuit (ASIC), or other programmed logic device. Logic may also be fully embodied as software.

The term “processor,” as used herein includes, but is not limited to, one or more of virtually any number of processor systems or stand-alone processors, such as microprocessors, microcontrollers, central processing units (CPUs), and digital signal processors (DSPs), in any combination. The processor may be associated with various other circuits that support operation of the processor, such as random-access memory (RAM), read-only memory (ROM), programmable read-only memory (PROM), erasable programmable read only memory (EPROM), clocks, decoders, memory controllers, or interrupt controllers, etc. These support circuits may be internal or external to the processor or its associated electronic packaging. The support circuits are in operative communication with the processor. The support circuits are not necessarily shown separate from the processor in block diagrams or other drawings.

The term “controller,” as used herein includes, but is not limited to, any circuit or device that coordinates and controls the operation of one or more input and/or output devices. A controller may, for example, include a device having one or more processors, microprocessors, or central processing units capable of being programmed to perform functions.

The term “logic,” as used herein includes, but is not limited to hardware, firmware, software, or combinations thereof to perform a function(s) or an action(s), or to cause a function or action from another element or component. Based on a certain application or need, logic may, for example, include a software controlled microprocess, discrete logic such as an application specific integrated circuit (ASIC), or other programmed logic device. Logic may also be fully embodied as software. As used herein, the term “logic” is considered synonymous with the term “circuit.”

The term “software,” as used herein includes, but is not limited to, one or more computer readable or executable instructions that cause a computer or other electronic device to perform functions, actions, or behave in a desired manner. The instructions may be embodied in various forms such as routines, algorithms, modules, or programs including separate applications or code from dynamically linked libraries. Software may also be implemented in various forms such as a stand-alone program, a function call, a servlet, an applet, instructions stored in a memory, part of an operating system or other type of executable instructions. It will be appreciated by one of ordinary skill in the art that the form of software is dependent on, for example, requirements of a desired application, the environment it runs on, or the desires of a designer/programmer or the like.

Augmented reality (AR) provides an enhanced version of the real, physical world using digital visual elements, sound, and/or other sensory stimuli which is delivered to a user via various technologies, Augmented reality adds or overlays visual, auditory, or other sensory information on real-world experiences to enhance one's experience. As set forth above, augmented reality can allow intra-operative visualization of complex pathology that can improve surgical efficiency while reducing surgical complications. Augmented reality in medicine is, for example, discussed in Zagury-Orly I, Solinski MA, Nguyen L H, Young M, Drozdowski V, Bain P A, Gantwerker E A. What is the Current State of Extended Reality Use in Otolaryngology Training? A Scoping Review. Laryngoscope. 2022 May 12. doi: 10.1002/lary.30174. Epub ahead of print. PMID: 35548939; Casari F A, Navab N, Hruby L A, Kriechling P, Nakamura R, Tori R, de Lourdes Dos Santos Nunes F, Queiroz M C, Fürnstahl P, Farshad M. Augmented Reality in Orthopedic Surgery Is Emerging from Proof of Concept Towards Clinical Studies: a Literature Review Explaining the Technology and Current State of the Art. Curr Rev Musculoskelet Med. 2021 April; 14(2):192-203. doi: 10.1007/s12178-021-09699-3. Epub 2021 Feb. 5. PMID: 33544367; PMCID: PMC7990993; Jud L, Fotouhi J, Andronic O, Aichmair A, Osgood G, Navab N. Farshad M. Applicability of augmented reality in orthopedic surgery—A systematic review. BMC Musculoskelet Disord. 2020 Feb. 15;21(1):103. doi: 10.1186/s12891-020-3110-2. PMID: 32061248; PMCID: PMC7023780; Bollen E, Awad L, Langridge B, Butler P E M. The intraoperative use of augmented and mixed reality technology to improve surgical outcomes: A systematic review. Int J Med Robot. 2022 Aug. 15:e2450. doi: 10.1002/rcs.2450. Epub ahead of print. PMID: 35971649; Guha D, Alotaibi NM, Nguyen N, Gupta S, McFaul C, Yang V X D. Augmented Reality in Neurosurgery: A Review of Current Concepts and Emerging Applications. Can J Neurol Sci. 2017 May;44(3):235-245. doi: 10.1017/cjn.2016.443. Epub 2017 Apr. 24. PMID: 28434425; Meola A, Cutolo F; Carbone M, Cagnazzo F, Ferrari M, Ferrari V. Augmented reality in neurosurgery: a systematic review. Neurosurg Rev. 2017 October;40(4):537-548. doi: 10.1007/s10143-016-0732-9. Epub 2016 May 7. PMID: 27154018; PMCID: PMC6155988; Cho J, Rahimpour S, Cutler A, Goodwin C R, Lad S P, Codd P. Enhancing Reality: A Systematic Review of Augmented Reality in Neuronavigation and Education. World Neurosurg. 2020 July;139:186-195. doi: 10.1016/j.wneu.2020.04.043. Epub 2020 Apr. 18. PMID: 32311561; and Sayadi L R, Naides A, Eng M, Fijany A, Chopan M, Sayadi J J, Shaterian A, Banyard D A, Evans G R D, Vyas R, Widgerow A D. The New Frontier: A Review of Augmented Reality and Virtual Reality in Plastic Surgery. Aesthet Surg J. 2019 Aug. 22;39(9):1007-1016. doi: 10.1093/asj/sjz043. PMID: 30753313.

Existing augmented reality headsets or headset systems lack functionality desirable for use in intra-operative visualization. In that regard, such devices or systems lack a number of surgery-specific adaptations. As a result, use of such devices or systems is limited and often cumbersome, which results in barriers to adoption in the medical field/surgery. In the case of currently available headsets, a surgeon may, for example, need to remove equipment such as surgical loupes and one or more headlights to enable donning of an augmented-reality headset. Loupes are small magnification devices used to see details more clearly. To use the loupes or headlights after donning such a headset, the surgeon would have to remove the headset. Such practices are quite cumbersome and represent a significant obstacle to adoption of augmented-reality headsets in the medical field and particularly in surgery.

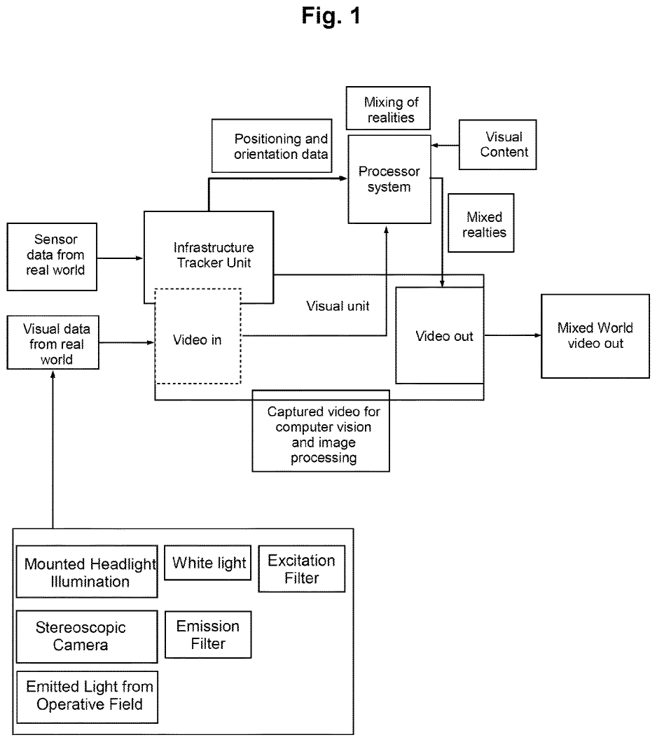

In a number of embodiments of augmented-reality headsets hereof, various functionalities or components used in the surgical and/or other medical theaters are incorporated into the headset. A typical architecture for augmented reality software upon which information from one or more embodiments of a headset system hereof is overlain is illustrated in FIG. 1. One or more such functionalities or components may be added to an existing headset as one or more dynamic add-ons. For example, one or more functionalities or components may be added to an existing headset through USB (for example, USB-C) connectivity. Additionally or alternatively, one or more such functionalities or components may be incorporated integrally into the architecture of the headset. By incorporating surgical and/or other medical functionalities into an augmented reality device (either as an add-on or integrally), AR information can be provided/streamed to the user and overlayed upon real-world information. while providing the functionality of equipment commonly used in, for example, a surgical theater in a single device/system. In general, the medical/surgical functionalities or components described herein can be incorporated into any augmented reality headset. In a number of embodiments, the augmented reality headset provides an optical see-through display and an interactive augmented reality, 3D generated overly.

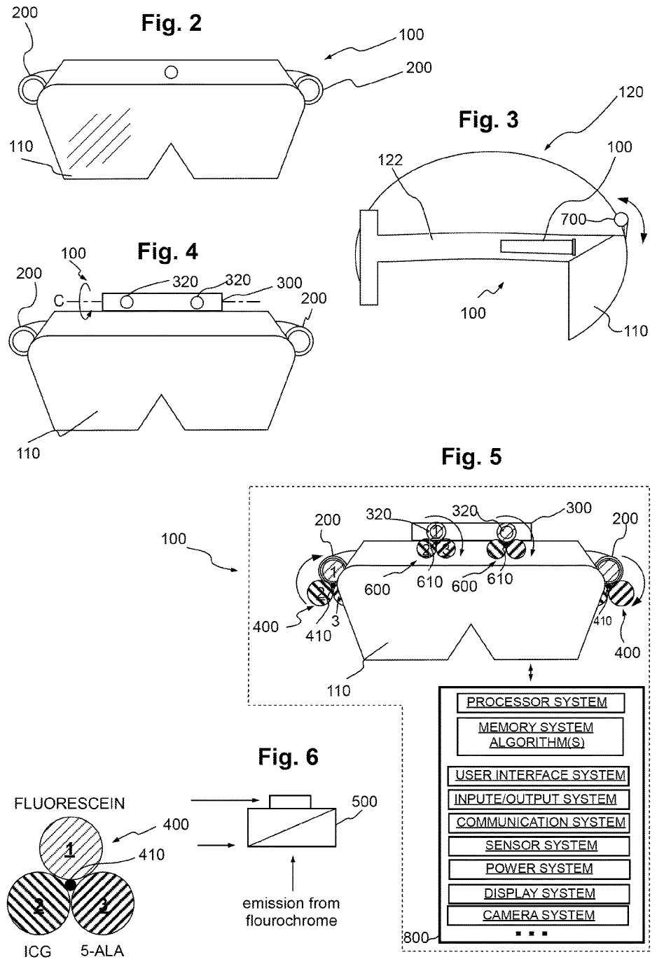

For example, a headset hereof may include one or more high-lumen headlights (that is, headlights having a luminous flux of at least 450 lumens) to illuminate at least a portion of the field of view of the user (for example, a surgical field). FIG. 2 illustrates an embodiment of an augmented reality headset or headset system 100 hereof including a head-mounted display 110 and one or more high-lumen headlights 200. As illustrated in FIG. 3, headlights 200 may, for example, be attached on each side to a laterally extending member of strap 122 of an adjustable support system 120 used to attach headset system 100 to a user's head. Bilateral headlights 200 (and/or other headlights hereof) may be controlled manually (that is, via hand controls, foot controls, etc.), via voice activation and/or via a projected virtual (for example, virtual holographic) menu.

Head-mounted display 110 in a number of embodiments is a see-through display to, for example, limit obscuring of a surgeon's view. Optical see-through displays may utilize a head-mounted display which, for example, uses optical combiners to merge images within an open-view head-mounted display. In the case of see-through augmented reality, lasers or LEDs may, for example, be used to project images onto an area of transparent glass, or into a user's eye, allowing the user to also see the real world directly. A number of displays used in augmented-reality headsets are ‘video passthrough’ displays in which image processing and graphics rendering blend and display physical and generated reality in real time. However, because of technological limitations, video passthrough wearable devices typically provide a field of view between 90 and 120 degrees, significantly limiting the user's peripheral vision. Basing head-mounted display 110 on optical, see-through technology results in significantly lower impact upon the user's physical world field of view. Using see-through displays in headsets hereof may, for example, result in more ready adoption of augmented reality in surgery. Augmented reality headsets including see-through display include the MICROSOFT® HOLOLENS@ 2 and HOLOLENS 3 (under development) AR headsets available from Microsoft, Corporation of Redmond, WA and the MAGIC LEAP® 2 AR headset available from Magic Leap of Plantation, Florida. The HOLOLENS 2, HOLOLENS 3 and MAGIC LEAP 2 systems are representative examples of systems that may be adapted for the user herein by adding on the functionalities or components described herein and/or by incorporating such functionalities or components into the architecture or design of the headset.

FIG. 4 illustrates an embodiment of headset 100 further including a three-dimensional (3D) camera 300 which may, for example, have an adjustable focal distance to allow for safe operation. An example of a 3D, stereoscopic camera which is readily adaptable for use in headset 100 is the ZED Mini camera available from StereoLabs of San Francisco, CA. Such cameras capture high-resolution stereo video for mixed-reality applications in 3D. Headlights 200 may be coordinated with stereoscopic camera 300 to provide constant illumination. The 3D camera may be controlled manually, via voice activation and/or via a virtual (for example, virtual holographic) menu.

3D camera 300 may, for example, provide varying degrees of magnification that are sufficient to provide the functionality of loupes. Magnification to provide the functionality of loupes may, for example, be in the range of 2.5 to 3.5×. Moreover, additional levels of magnification (for example, up to 40× in a number of embodiments) can be provided by camera 300 to provide at least some of the functions of a microscope. Such microscopes typically range in price from $200,000 to $800,000. In additions to the other benefits described herein, including microscope functionality in headset 100 can thus provide significant cost saving in certain circumstances. At higher magnification, motion stabilization functionality may be provided in a number of embodiments to improve stability in the visualization of the magnified field.

As set forth above, camera 300 may be adapted from an existing camera, such as the ZED Mini camera. That camera is designed to interface with virtual reality headsets. It slides over the external cameras of such devices. The output of the camera may, for example, be used as the primary video source and be integrated with headset 100 to have that video displayed as a planar hologram. The camera may be readily modified, if desired, to accommodate, for example, 40× zoom, which is the upper limit of surgical microscopes currently on the market. The camera may also readily be integrated with eye-tracking architecture in currently available headsets as a means of finer control while under high magnification. Headlights 200 may be operated in a coordinated manner with camera 300. Headlights 200 and camera 300 may, for example, be integrated with eye-tracking software such that the two units are controlled/move in concert to, for example, keep the operative field properly illuminated. Movement of camera 300, and by extension headlights 200, can also occur manually, by voice command or through a holographic menu as described above. Furthermore, manual adjustment is possible as is accomplished with current loupe and headlight systems. Dynamic focusing may be achieved by integration of camera 300 with the spontaneous localization and mapping system of the AR software of headset 100. When focus is set, that focal point may, for example, be locked in a coordinate system. The camera magnification and/or focus may be adapted with head movement to keep that coordinate (for example, x, y, z) in focus.

It is well documented in literature that surgery presents a healthcare risk to surgeons. In that regard, surgeons often sustain neck injuries as a result of continuous and repeated craning over patients, sometimes requiring surgical repair. Loupes have, for example, been developed at 45 degrees to maintain the surgeon's head in an upright neutral position. Camera 300 may likewise offer the ability to maintain the head of a surgeon in a neutral position while offering varying and high degrees of magnification to operate in the manner of, for example, loupes and microscopes. Camera 300 may, for example, be fixed in a particular positioned or adjustably pivotable about an axis C as illustrated in FIG. 4 over a range of angles relative to the orientation illustrated in FIG. 4. In the illustrated orientation, lenses 320 of camera 300 are oriented generally horizontally (that is, perpendicular to the gravitational field or perpendicular to the orientation of the page). In a number of embodiments. camera 300/camera lenses 320 is fixed or may be adjusted to a position in the range of 0 to 90° or in the range of 0 to 45° (toward the direction of the surgical field) relative to the illustrated orientation of FIG. 4. The angle of camera 300/camera lenses 320 may, for example, be adjustable via a motor which is represented schematically by the arced arrow in FIG. 4. Control of the angle of camera 300/camera lenses 320 may be achieved manually, via voice activation and/or via a virtual (for example, virtual holographic) menu as described above.

As illustrated schematically in FIG. 5, headset 100 can further include an excitation filter system 400 in operative connection with one or more of headlights 200. Filter systems 400 may, for example, include multiple filters and be manipulable or movable (for example, rotatable, slidable, etc.) to select one (or none) of the multiple filters for use in connection with headlight 400. In the illustrated embodiment, filter systems 400 include three filters (1, 2, and 3). Rotating excitation filter system 400 about its axis to align of the filters thereof with headlight(s) 200 may be used to visualize important intra-operative fluorescent/fluorophoric molecules such as fluorescein (filter 1), indocyanine green (ICG; filter 2), and 5-aminoluvelinic acid (5-ALA; filter 3) as illustrated in FIG. 6 for filters 1, 2, and 3 respectively. When placed in operative connection with a headlight 200, filter system 400 may, for example, be rotated to align one of filters 1, 2, and 3 with headlight 200 using a small motor 410 as illustrated schematically in FIGS. 5 and 6. If one of headlights 200 has a sufficiently high luminous flux, only a single excitation filter system 400 may be required in connection with that headlight 200. However, an excitation filter system 400 may be provided in operative/optical connection with each headlight 200 of headset 100.

Excitation filter system 400 may be combined with a dichroic mirror 500, in certain embodiments (see FIG. 6; as known in the microscope arts), and with an emission filter system 600 in operative/optical connection with each lens 320 of camera 300 as illustrated in FIG. 5. Similar to excitation filter systems 400, each of emission filter systems 600 may include multiple filters and be manipulable (for example, rotatable, slidable, etc.) to select one (or none) of the multiple filters for use in connection with lens 320. In the illustrated embodiment, emission filter systems 600 include three filters (1, 2, and 3) such as fluorescein (filter 1), indocyanine green (ICG; filter 2), and 5-aminoluvelinic acid (5-ALA; filter 3) as described above in connection with filter system 400. Emission filter system 600 may, for example, be rotated to align one of filters 1, 2, and 3 with lens 320 using a small motor 610 as illustrated schematically in FIGS. 5 and 6. Control of excitation filter system 400 and emission filter system 600 may be achieved manually, via voice activation and/or via a virtual (for example, a virtual holographic) menu. Currently, multiple-filter systems such as excitation filter system 400 and emission filter system 600 are available only on microscopes. Such filter systems are, for example, important for aneurysm surgery and primary brain tumor surgery.

As appreciated by those of skill in the art, one of filters 1, 2 or 3 filter systems 400 and 600 hereof will not always be in operative connection with the headlight(s) 200 and camera lenses 300. Each of filter systems 400 and 600 may, for example, be movable so that a filter of the filter system is not aligned with one of headlight 200/lens 320. There may, for example, be a void space or a unit with transparent/non-filtering glass among the filters of a filter system that is rotatable about an axis. Each filter of a filter system may, alternatively, be individually slidable into and out of operative connection with one of headlights 200/lenses 320.

For certain headsets in which head-mounted, heads-up display 110 is adjustable, an additional feature that mechanically raises display 110 up and lowers (or otherwise moves/positions) it in place may be provided for surgeon comfort when, for example, looking at physical screens in the operating room (for example, a physical screen attached to a C-arm fluoroscopy system). A device 700 to adjust the position of display 110 is illustrated schematically in FIG. 3. Device 700 may, for example, be operable to pivot or rotate display 110 upward and downward as illustrated by the arced arrow in FIG. 3. Device 700 may be controlled manually, via voice activation and/or via a virtual (for example, a virtual holographic) menu.

As illustrated schematically in FIG. 5, headset 100 includes a control system 800 which may be attached to or otherwise be in operative connection with headset 100. Control system 800 may, for example, include a processor system. The processor system may, for example, include one or more processors such as microprocessors as known in the art. A memory system is in operative/communicative connection with the processor system. Readable instructions (software components) are stored on the memory system and are executable by the processor system. A user interface system is also provided in operative/communicative connection with the processor system (including a display/display generation system as known in the computer/AR arts). A sensor system may be provided to including one or more sensor to sense one or more variables. At least partial control of one or more components of headset 100 may be achieved (for example, via feedback control) on the basis of sensing one or more variables. Control system 800 may further include a communication system in operative connection with the processor system and a power system (for example, a battery array) to power components of control system 800 as well as other components of headset 100. The communication system may, for example, include a microphone for effecting voice activation of various components of headset 100 as described above.

Processing of data may, for example, occur only in a processor system attached to headset 100 or may be distributed among a plurality of processor systems. One or more such processor systems may be remote from headset 100. In addition to or as part of software to display an augmented-reality reality overlay, the memory system may further have stored therein streaming software to provide operation of camera 300 with sufficiently low enough latency so that there is minimal noticeable delay between what is being represented holographically in the surgical field and what is actually happening in the surgical field, thereby enabling the surgeon to operate fluidly.

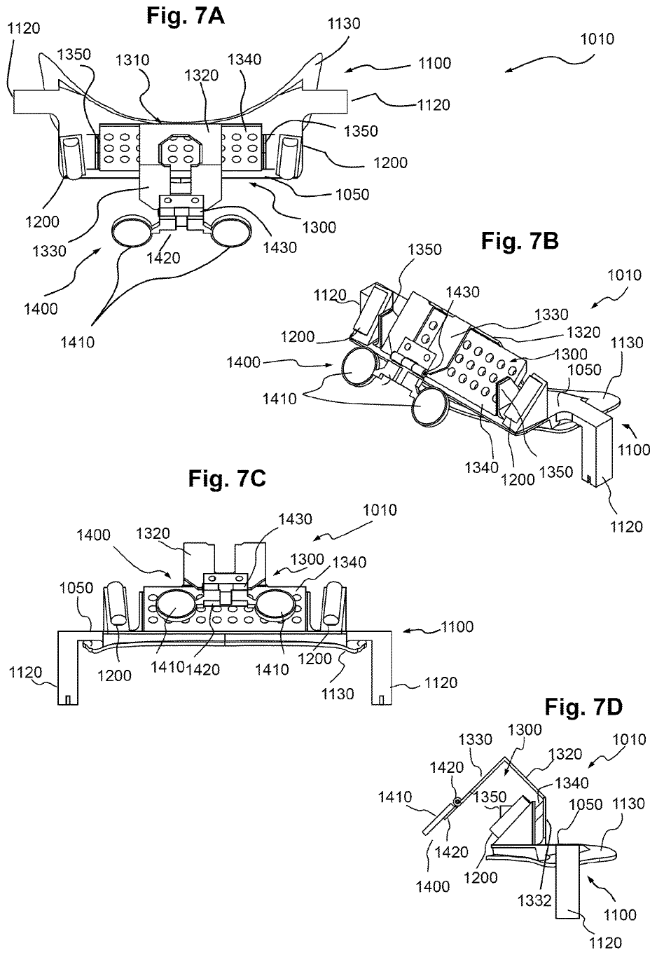

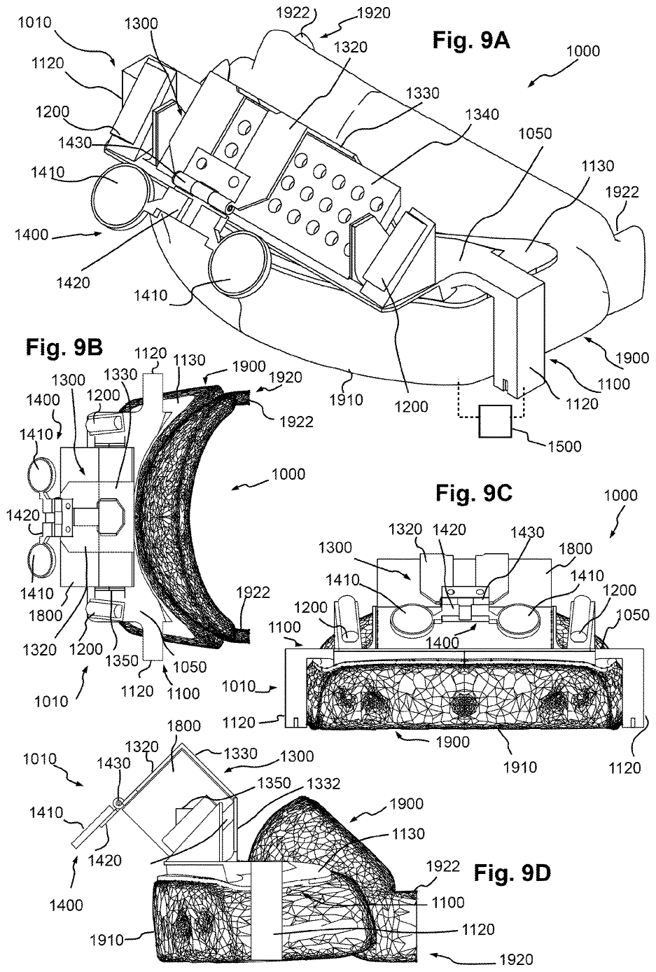

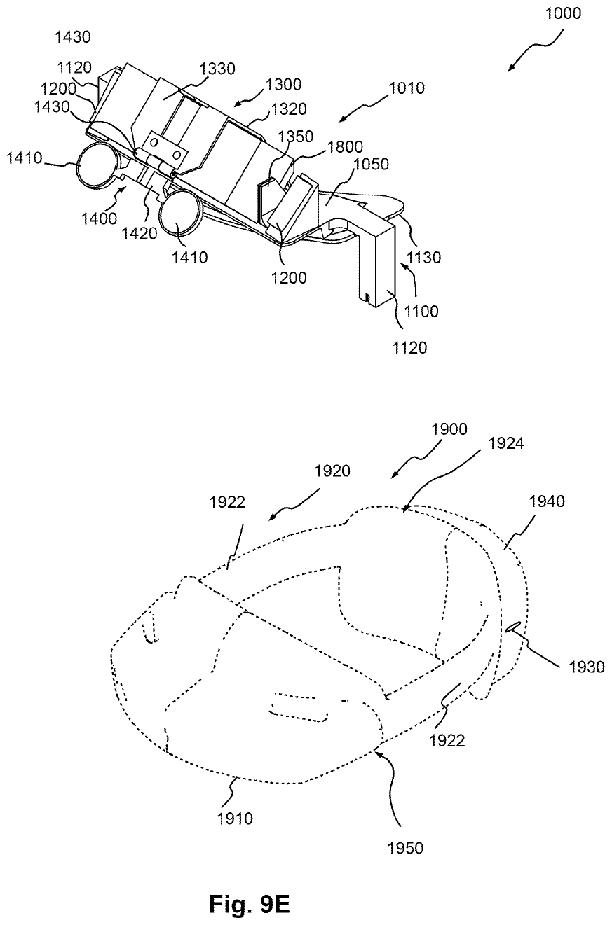

As discussed above, one or more such functionalities or components as described above may be added to an existing augmented reality headset as one or more dynamic add-ons. In that regard, FIGS. 7A through 9D illustrates a representative embodiment of a device or system 1010 hereof via which one or more such functionalities can be added to a separately manufactured augmented reality headset such as the HOLOLENS 2 augmented reality headset 1900. In the embodiment of FIGS. 7A through 9D, device 1010 is configured for use in connection with a separately manufactured stereoscopic camera 1800 and a separately manufactured augmented reality headset 1900 to form an augmented or mixed reality system 1000 (see, for example, FIGS. 9A through 9E), Device 1010 thus functions as an attachment or add-on for currently available and future augmented reality (AR) headsets such as augmented reality headset 1900 and provides additional functionality to augmented reality headset 1900 as described above. In that regard, operative attachment of device provides utility to multiple surgical specialties in the operating room as well as other similar environments such as the primary cares physician's office and the dentist's office. In a number of embodiments, attachment device 1010 adds, for example, three functionalities to augmented reality headset 1900 in forming augmented reality system 1000: a stereoscopic camera, high-powered surgical lights with multiple emission wavelengths, and light filters to modify the wavelengths of light the doctor can visualize. Those features come together in an integrated system 1000 to, for example, provide surgeons the tools/functionalities they utilize during surgery in addition to the enhancing capabilities of AR.

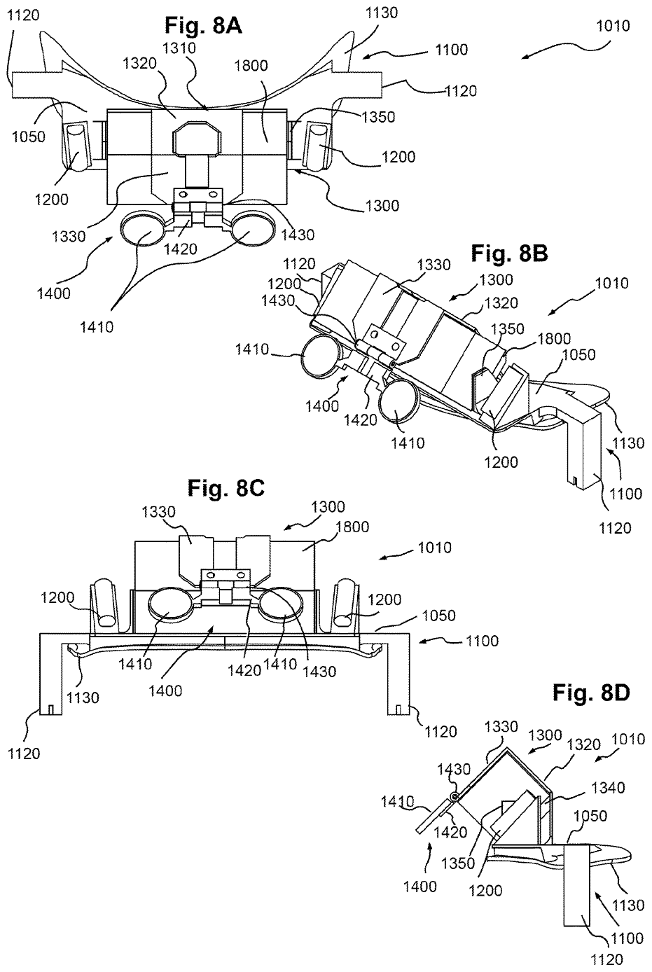

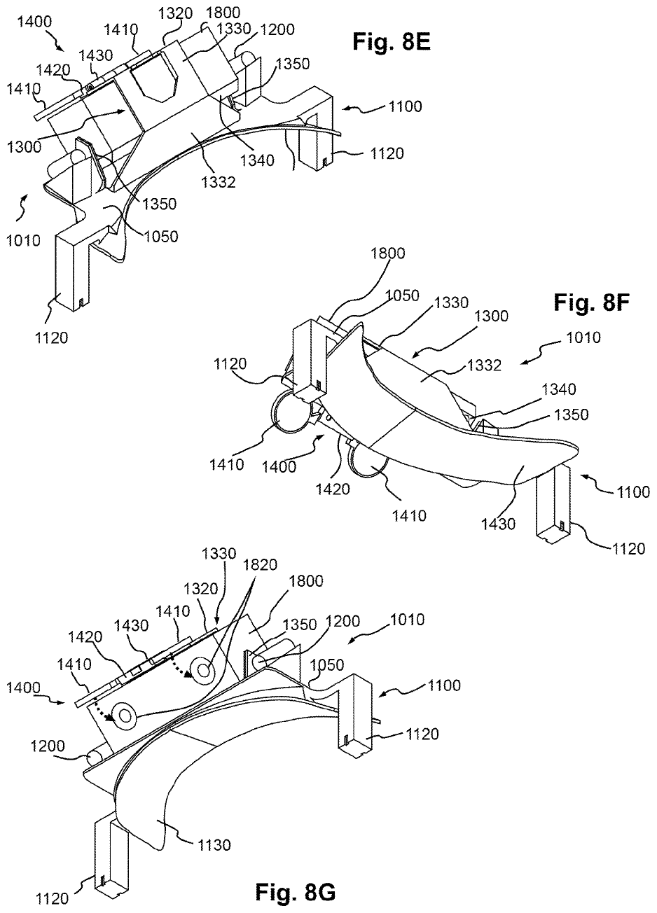

In FIGS. 7A through 7D, device 1010 is illustrated without a camera 1800 or augmented reality headset 1900 in operative connection therewith or attached thereto. In FIGS. 8A through 8G, device 1010 is illustrated with camera 1800 in operative connection therewith or attached thereto. In FIGS. 9A through 9D, device 1010 is illustrated with camera 1800 and augmented reality headset 1900 in operative connection therewith or attached thereto.

Referring, for example, to FIGS. 7A through 7D, device 1010 includes a support, body, or frame 1050 having attached thereto (or partially or completely integrated therewith or formed thereby) an interface 1100 to interface with and form a connection or operative connection with augmented reality headset 1900. Interface 1100 may be designed specifically to interface with a particular augmented reality headset 1900 or may be operable to interface with a plurality of different augmented reality headsets (for example, via adjustment of one or more components of interface 1100). In the illustrated embodiment, interface 1100 includes extending members or bracket members 1120 which extend from each side of body 1050 and interact with sides of augmented reality headset 1990. Extending members 1120 may, for example, be adjustable in length as well as in position to change the distance or width therebetween to interact with augmented reality headset 1900. Interface 1100 further includes a base or support 1130 which, in the illustrated embodiment, includes a lower surface which is contoured to generally conform to the contour of a portion of an upper surface of reality headset 1900.

Device 1010 further includes one or more lights or headlights 1200. In the illustrated embodiment, one light 1200 is attached to each side of body 1050. Lights 1200 function as described in connection with lights 200. Light 1200 may, for example, be high powered surgical lights with multiple emission wavelengths. High density surgical lights may, for example, be positioned on both lateral sides of camera 1800. Lights 1200, which may be angled down at (for example, at 45 degrees), are functional to illuminate the field in front of the surgeon or other user. In a number of embodiments, the user is also able to customize their lighting preferences with multiple light settings and the ability to control the angle of lights 1200. The user may, for example, make such adjustments through voice commands as described above, through an interactive AR menu, and/or via eye tracking. In a number of embodiments, lights 1200 have white light capabilities as well as other commonly used wavelengths such as UV light. The ability to change wavelengths of light is, for example, important for a user's ability to conduct a fluorescence guided surgery or FGS procedure.

Device 1010 further includes a camera interface 1300 to form a connection/operative connection with separately manufactured camera 1800 (see, for example, FIGS. 8A through 8G). In the illustrated embodiment, camera interface 1300 includes a retention or clamp system 1310 which includes a first or rearward member 1320 which contacts and cooperates with a first side or rearward side of camera 1800 and a second member 1330 (angled approximately 90 degrees with respect to first member 1320 in the illustrated embodiment) which contacts and cooperates with a second or upper side of camera 1800. In the illustrated embodiment, second member 1330 extends from an extending member 1332 which extends from body 1050 (see FIG. 7D, for example). Camera interface 1300 further includes a lower or base member 1340 which contacts and cooperates with a third or lower side of camera 1800. A plane of base member 1340 may, for example, be oriented generally parallel to a plane of second or upper member 1330. In the illustrated embodiment, camera 1800 thus rests on the base of the attachment and is secured by clamp system 1310 that extends along the rearward and the upper side of cameral 1800. Side or lateral members 1350 may, for example, be provided on each lateral side of base member 1340 to assist in retaining stereoscopic camera 1800 in connection with device 1010. Camera interface 1300 may, for example, be formed as a bracket which is attached to body 1050.

In a number of embodiments, when camera 1800 is attached into camera interface 1300, stereoscopic camera 1800 is latched into camera interface 1300 and may be angled down at, for example, 45 degrees to provide the best viewing angle for a user such as a surgeon. Camera 1800 may be connected directly into the augmented reality headset 1900 to provide the user with a display of the video feed in augmented reality headset 1900. The user may be provided with the ability to control magnification of camera 1800 (to zoom in and zoom out) with the use of voice commands, an interactive AR menu, and/or eye tracking. As described above, camera 1800 may thus operate as “virtual” loupes for a user such as a doctor/surgeon. The user may, for example, be able to customize their visual preferences and adjust the viewing angle of camera 1800 through a hands-free mechanical system controlled through augmented reality headset 1900.

In the illustrated embodiment, retention or clamp system 1300 is connected to a set of light filters that are operable to flip into optical alignment with camera lenses 1820 (see FIG. 8G). Device 1010 further includes a filter system or emission filter systems 1400 which may include multiple or tunable filters 1410 similar in function to filter system 400 described above. One or more filters 1410 may, for example, be interchangeable or tunable to accommodate multiple light spectrums. Emission filter system 1400 may, for example, include a filter support 1420 which may be movable, for example, rotated about an axis of a hinge mechanism 1430, which connects to support 1420 for filters 1410 to bring filters 1420 into optical alignment with lens 1820 of camera 1800. Such movement may be manually actuated or actuated in a powered manner using an actuator such as a small motor (not shown). By placing filters 1410 directly in front of the camera lens, the camera will be able to visualize, for example, any fluorescent light invisible to the naked eye and project this image to the doctor. Filters 1410 may, for example, be raised and lowered through a hands-free mechanical system controlled through the AR headset. Filters 1410 are, for example, essential for a doctor's ability to conduct an FGS procedure. As described above 3D stereoscopic camera 1800, headlights 1200, emission filter system 1400, and any positioning system may, for example, be placed in operative connectivity with augmented reality headset 1900 via, for example, a connection or communication system 150 (illustrated schematically in FIG. 9A), which may, for example, include a USB or other electrical/data connections as known in the electrical and computer arts.

The embodiment of device 1010 illustrated in FIGS. 7A through 9E is illustrated to be compatible with the MICROSOFT HOLOLENS 2 augmented reality headset 1900. The design of device 1010 is such that connecting device 1010 to headset 1900 does not interfere with controls of the headset (for example, control components to adjust, brightness, volume, etc.). As, for example, illustrated in FIG. 9E, augmented reality headset includes a rotatable and see-through visor/display 1910. A band or arm system 1920 including arms or bands 1922, which connect to a rear pad 1924, is used to retain augmented reality headset 1900 on the head of a user. An upper strap (not shown) may also be used to cooperate with the top of a user's head. A USB port 1930 may, for example, be provided in a rear component housing 1940a. System electronics/components may, for example, be distributed between a front component housing/support 1950 and rear component housing 1940. The components of device 1010 can readily be arranged to be compatible with an augmented reality headset by, for example, easily adjusting or modifying interface 1100 to a particular augmented reality headset. An attachment mechanism 1127 (illustrated schematically in FIG. 9E) may, for example, be provided to aid in secure attachment of device 1010 to augmented reality headset 1900. Additionally, camera interface 1300 can be readily adjusted or modified to fit the dimensions of different stereoscopic cameras. Likewise, device 1010 is readily adjustable or modifiable to be used in connection with a wide variety of lights or headlights 1200. As known in the art, an external power source (not illustrated) may be used to charge/power the augmented reality headset (which typically includes a rechargeable battery system) through its native power input.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

1. A device for use in connection with an augmented reality headset as an add-on component to the augmented reality headset, the augmented reality headset including a support system for supporting the headset upon the head of a user, a display system attached to the support system and comprising a display, and a control system comprising a processor system and a memory system in communicative connection with the processor system, the memory system having stored therein one or more software algorithms executable by the processor system, the control system being configured to provide an augmented reality display via the display system, comprising:
a body formed separately from the augmented reality headset;
a headset interface system attached to the body to physically connect the device to the augmented reality headset; and
at least one of a 3D stereoscopic camera, a headlight, an excitation filter system, and an emission filter system being attached to the body; and
a communication interface system attached to the body and configured to place one or more of the at least one of a 3D stereoscopic camera, the headlight, the excitation filter system, and the emission filter system being attached to the body in communicative connection with the control system of the augmented reality headset.

2. The device of claim 2 wherein the body comprises a 3D stereoscopic camera which is connected to the body via a camera interface attached to the body, the camera interface being configured to connect the 3D stereoscopic camera thereto such that the 3D stereoscopic camera is orientable to provide a view of at least a portion of a field of view of the user when the device is in connection with the augmented reality headset.

3. The device of claim 1 wherein a plurality of headlights are attached to the body, each of the plurality of headlights providing at least 450 lumens of light.

4. (canceled)

5. The device of claim 3 wherein at least one of: (i) the plurality of headlights is configured to controllably emit light of different ranges of wavelengths, and (ii) the device further comprises the excitation filter system attached to the body and being controllably positionable to be in optical connection with one of the one or more headlights.

6. The device of claim 5 wherein at least one of the ranges of wavelengths is suitable to enhance the visualization of fluorophoric molecules or the excitation filter system is configured to enhance the visualization of fluorophoric molecules.

7. The device of claim 5 wherein the device comprises an excitation filter system and the excitation filter system comprises at least one filter for each of fluorescein, indocyanine green, and 5-aminoluvelinic acid.

8. The device of claim 2 further comprising an emission filter system attached to the augmented reality headset and being controllably positionable to be in operative connection with one of one or more lenses of the 3D stereoscopic camera.

9. The device of claim 8 wherein the emission filter system is configured to enhance the visualization of fluorophoric molecules.

10. The device of claim 9 wherein the emission filter system comprises multiple filters.

11. The device of claim 10 wherein the emission filter system comprises at least one filter for each of fluorescein, indocyanine green, and 5-aminoluvelinic acid.

12. The device of claim 1 wherein at least one of the 3D stereoscopic camera, the headlight, the excitation filter system, when present, and the emission filter system, is controllable via at least one of manual control, voice activation or a menu of the augmented reality headset, optionally a holographic menu.

13. The device of claim 1 wherein each of the 3D stereoscopic camera, the headlight, the excitation filter system, when present, and the emission filter system, is controllable via at least one of manual control, voice activation or a menu of the augmented reality headset, optionally a holographic menu.

14. The device of claim 2 wherein the 3D stereoscopic camera and the headlight are controllable in concert.

15. (canceled)

16. A system, comprising:
an augmented reality headset comprising a support system for supporting the augmented reality headset upon the head of a user, a display system attached to the support system and comprising a display, a control system comprising a processor system and a memory system in communicative connection with the processor system, the memory system having stored therein one or more software algorithms executable by the processor system, the control system being configured to provide an augmented reality display via the display system; and
a device for use in connection with an augmented reality headset as an add-on component to the augmented reality head set, the device comprising a body formed separately from the augmented reality headset, a headset interface system attached to the body to physically connect the device to the augmented reality headset; at least one of a 3D stereoscopic camera, a headlight, an excitation filter system, and an emission filter system being attached to the body; and a communication interface system attached to the body and configured to place one or more of the at least one of a 3D stereoscopic camera, the headlight, the excitation filter system, and the emission filter system being attached to the body in communicative connection with the control system of the augmented reality head set.

17. The system of claim 16 wherein the body comprises a 3D stereoscopic camera which is connected to the body via a camera interface attached to the body, the camera interface being configured to connect the 3D stereoscopic camera thereto such that the 3D stereoscopic camera is orientable to provide a view of at least a portion of a field of view of the user when the device is in connection with the augmented reality headset.

18. The system of claim 17 wherein a plurality of headlights are attached to the support system, each of the plurality of headlights providing at least 450 lumens of light.

19. (canceled)

20. The system of claim 17 wherein at least one of: (i) the headlight is configured to controllably emit light of different ranges of wavelengths, and (ii) the system further comprises the excitation filter system attached to the headset and being controllably positionable to be in optical connection with one of the one or more headlights.

21. The system of claim 20 wherein at least one of the ranges of wavelengths is suitable to enhance the visualization of fluorophoric molecules or at least one the excitation filter system is configured to enhance the visualization of fluorophoric molecules.

22.-27. (canceled)

28. The system of claim 16 wherein at least one of the 3D stereoscopic camera, the headlight, the excitation filter system, and the emission filter system is controllable via voice activation or a holographic menu of the augmented reality headset.

29.-30. (canceled)

31. The system of claim 17 wherein the 3D stereoscopic camera and the headlight are controllable in concert.

32.-33. (canceled)

34. The system of claim 16 wherein the communication interface system comprises a USB connection.

35. (canceled)

36. A method of adapting an augmented reality headset for use in a medical procedure, comprising:
providing a device which is formed separately from the augmented reality headset, the device comprising a body formed separately from the augmented reality headset, a headset interface system attached to the body to physically connect the device to the augmented reality headset; at least one of a 3D stereoscopic camera, a headlight, an excitation filter system, and an emission filter system being attached to the body; and a communication interface system attached to the body and configured to place one or more of the at least one of a 3D stereoscopic camera, the headlight, the excitation filter system, and the emission filter system being attached to the body in communicative connection with a control system of the augmented reality head set; and
attaching the device to the augmented reality headset via the headset interface system.

37.-44. (canceled)
